SEQUENCE LISTING (4 sequences)

SEQ ID NO: 1 - DNA, 1453 bp, homo sapiens
aagcttgggctgcaggtcgatcgactctagaggatcgatccccgggcgagctcgaattcgcaaccaccatggtcagctactgggacaccggggtcctgctgtgcgcgctgctcagctgtctgcttctcacaggatctagttccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaagggcccgggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagcggccgc

SEQ ID NO: 2 - protein, 458 aa, homo sapiens
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGPGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 1377 bp, homo sapiens
atggtcagctactgggacaccggggtcctgctgtgcgcgctgctcagctgtctgcttctcacaggatctagttccggaagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 458 aa, homo sapiens
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2005/0196340 A1
(43) Pub. Date: Sep. 8, 2005

(54) Use of a VEGF antagonist in combination with radiation therapy

(76) Inventors: Jocelyn Holash, Alameda, CA (US); George Yancopoulos, Yorktown Heights, NY (US); Phyllis R. Wachsberger, Berwyn, PA (US); Adam P. Dicker, Cherry Hill, NJ (US); Randy Burd, Wayne, PA (US)

Correspondence Address: Valeta Gregg, Esq.;Regeneron Pharmaceuticals, Inc., 777 Old Saw Mill River Road, Tarrytown, NY, 10591, US

(21) Appl. No.: 10/998,881

(22) Filed: Nov. 29, 2004

Related U.S. Application Data
(63) Continuation-in-part of application No. 10/909,011, filed on Jul. 30, 2004.
(60) Provisional application No. 60/492,864, filed on Aug. 6, 2003.

(51) Int. Cl. A61K 38/17; A61K 51/00

(52) U.S. Cl. 424/1.69; 514/12

(57) ABSTRACT

Methods of treating cancer and/or reducing or inhibiting tumor growth in a subject in need thereof, comprising administering pharmaceutical composition comprising a vascular endothelial cell growth factor (VEGF) antagonist, in combination with radiation therapy and/or a therapeutic radiopharmaceutical.

### CROSS-REFERENCE TO RELATED APPLICATION

This application is a continuation-in-part of U.S. Ser. No. 10/909,011 filed 30 Jul. 2004 and claims the benefit under 35 USC § 119(e) of U.S. Provisional application 60/492,864 filed 6 Aug. 2003, which application is herein specifically incorporated by reference in its entirety.

### BACKGROUND

1. Field of the Invention

The field of the invention is related to methods of treating cancer in a mammal with a vascular endothelial growth factor (VEGF) trap capable of binding and inhibiting the biological activity of VEGF in combination with radiation therapy.

2. Description of Related Art

Vascular endothelial growth factor (VEGF) has been recognized as a primary stimulus of angiogenesis in pathological conditions. Approaches to methods of blocking VEGF include soluble receptor constructs, antisense molecules, RNA aptamers, and antibodies. See, for example, PCT WO/0075319, for a description of VEGF-receptor based trap antagonists.

Radiation therapy is widely used for the treatment of cancer both alone and in conjunction with surgery and/or anti-neoplastic agents. Combination therapies using radiation and squalamine are known (see U.S. Pat. No. 6,596,712). Recent preclinical studies have suggested that radiation therapy in combination with VEGF targeting agents can enhance the therapeutic ratio of ionizing radiation by targeting both tumor cells and tumor vessels.

### BRIEF SUMMARY OF THE INVENTION

The invention is based in part on the results of experiments described below that show that the combined treatment of a VEGF trap with radiation therapy results in a significant inhibition of tumor growth in a clinically relevant human glioblastoma model.

Thus, in a first aspect, the invention features a method of treating cancer in a subject in need thereof, comprising administering to the subject a VEGF antagonist in combination with radiation therapy such that the cancer is treated, wherein the radiation therapy is administered as a single dose, a fractionated dose, or in multiple doses. In one embodiment of the invention, the VEGF antagonist is a VEGF trap, for example, Flt1D2.Flk1D3.FcΔC1(a) (SEQ ID NOs:1-2), or VEGFR1R2-FcΔC1(a) (SEQ ID NOs:3-4).

In specific embodiments, the amount of VEGF trap administered to a human subject is a low dose, for example about 1-5 mg/kg. In a more specific embodiment, a low dose is about 2.5-5 mg/kg. In another embodiment, the amount of VEGF trap administered to a human subject is a high dose, for example, about 7.5-15 mg/kg. In a more specific embodiment, a high dose is about 8-12 mg/kg. Administration may be by any method known in the art, including subcutaneous, intramuscular, intradermal, intraperitoneal, intravenous, intranasal, epidural, or oral. Preferably, administration is subcutaneous or intravenous, or a combination thereof. Administration may be concurrently (e.g., simultaneous) with, or sequentially (e.g., prior to or following radiation administration). In one embodiment, a low dose (≦5.0 mg/kg) of VEGF trap is administered concurrently with radiation once per week or at 2-4 week intervals. In another embodiment, a high dose (≧7.5 mg/kg) is administered with radiation once per month or at 2-4 month intervals. In another embodiment, a high dose of VEGF trap is administered concurrently with radiation once per week or at 2-4 week intervals. In another embodiment, a low dose is administered with radiation once per month or at 2-4 month intervals. In any embodiment of the invention, radiation therapy may be administered as a fractionated dose.

Radiation therapy, including therapeutic radiopharmaceuticals, can be administered to the mammal according to protocols commonly employed in the art and known to the skilled artisan. Such therapy may include cesium, iridium, iodine, or cobalt radiation. In one embodiment, the radiation therapy is ionizing radiation therapy.

In a second aspect, the invention features a method of reducing or inhibiting tumor growth in a subject in need thereof, comprising administering to the subject a vascular endothelial growth factor (VEGF) antagonist in combination with radiation therapy such that tumor growth is reduced or inhibited, wherein the radiation therapy is administered as a single dose, a fractionated dose, or in multiple doses.

In a third aspect, the invention features a method of treating a human patient suffering from cancer, comprising administering an effective amount of a vascular endothelial growth factor (VEGF) trap and radiation to the human patient, the method comprising administering to the patient an initial dose of ≦5.0 mg/kg of the VEGF trap with radiation therapy. In specific embodiments, the initial administration of VEGF trap and radiation are followed by a plurality of subsequent doses of the VEGF trap and radiation in an amount that is approximately the same or less of the initial dose, wherein the subsequent doses are separated in time from each other by at least one week.

Other objects and advantages will become apparent from a review of the ensuing detailed description.

### BRIEF DESCRIPTION OF THE FIGURE

FIG. 1 is a bar graph showing the effects of fractionated irradiation and high and low dose VEGF trap on tumor growth.

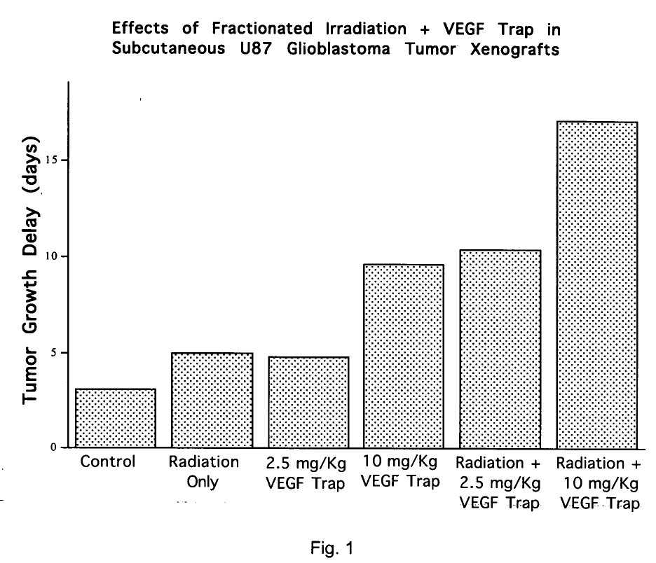

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

General Description

The invention is based on the findings that administration of a VEGF trap capable of binding and inhibiting the biological activity of VEGF, for example the VEGF trap VEGFR1R2-FcΔC1(a) (SEQ ID NOs:3-4), in combination with radiation therapy (including ionizing radiation and/or therapeutic radiopharmaceuticals, results in a significant inhibition of tumor growth. The effect of the combination of a VEGF trap and radiation therapy on tumor growth provides a promising therapeutic approach to the treatment of human cancer. For a description of VEGF-receptor-based antagonist VEGF traps Flt1D2.Flk1D3.FcΔC1(a) (SEQ ID NOs:1-2) and VEGFR1R2-FcΔC1(a) (SEQ ID NOs:3-4), see PCT WO/0075319, the contents of which is incorporated in its entirety herein by reference.

Methods of Administration

The invention provides methods of treatment comprising administering to a subject an effective amount of a pharmaceutical composition comprising a VEGF trap, in combination with radiation therapy. Various delivery systems are known and can be used to administer the composition of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, intraocular, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Administration can be acute or chronic (e.g. daily, weekly, monthly, etc.) or in combination with other agents. Pulmonary administration can also be employed, e.g., by use of an inhaler or nebulizer, and formulation with an aerosolizing agent.

In another embodiment, the active agent can be delivered in a vesicle, in particular a liposome, in a controlled release system, or in a pump. In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), by direct injection, or by use of microparticle bombardment, or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as silastic membranes, fibers, or commercial skin substitutes.

A composition useful in practicing the methods of the invention may be a liquid comprising an agent of the invention in solution, in suspension, or both. The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form. of an ointment.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention is preferably viscous or muco-adhesive, or even more preferably, both viscous and mucoadhesive.

Radiation Therapy and Therapeutic Radiopharmaceuticals

Radiation is used as a therapeutic treatment for many types of cancers and is delivered in various ways, depending on the disease, its location, and its stage. Such therapy may include cesium, iridium, iodine, or cobalt radiation. The radiation therapy may be whole body irradiation, or may be directed locally to a specific site or tissue in or on the body. Typically, radiation therapy is administered in pulses over a period of time from about 1 to about 2 weeks. The radiation therapy may, however, be administered over longer periods of time. Optionally, the radiation therapy may be administered as a single dose or as multiple, sequential doses. Examples of radiation therapies include conformal radiation therapy, coronary artery brachytherapy, fast neutron radiotherapy, intensity modulated radiotherapy (IMRT), interoperative radiotherapy, interstitial brachytherapy, interstitial breast brachytherapy, organ preservation therapy, and steriotactic radiosurgery. The use of therapeutic radiopharmaceuticals is also encompassed by the invention. Examples of therapeutic radiopharmaceuticals include, for example, P32 chromic phosphate colloid, P32 sodium chromate, Sr89 chloride, Sm153 EDTMP lexidronam, l131 sodium iodide, Y90 ibritumomab tiuxetan, In111 tositumomab, and Y90 microspheres. The VEGF trap is administered to the patient concurrently or sequentially of treatment with radiation and/or a therapeutic radiopharmaceutical compound. Following administration of the VEGF trap and radiation, the patient's cancer and physiological condition can be monitored in various ways well known to the skilled practitioner. For instance, tumor mass may be observed physically, by biopsy or by standard x-ray imaging techniques.

Pharmaceutical Compositions

The present invention provides pharmaceutical compositions comprising a VEGF trap and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin.

The composition of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the composition of the invention that will be effective for its intended therapeutic use can be determined by standard clinical techniques based on the present description. In addition, in vitro assays may optionally be employed to help identify optimal dosage ranges. Effective doses may be extrapolated from dose-response curves derived from in vitro or animal model test systems.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable. The therapy may be provided alone or in combination with other drugs.

Specific Embodiments

Example 1 describes experiments in which tumors grown in mice from U-87 glioblatoma cells were treated with a combination of low or high doses of the VEGF trap of SEQ ID NOs:3-4 with or without a single or fractionated dose of radiation. The results showed enhanced suppression and delay of tumor growth with the combination of VEGF inhibitor and radiation therapy.

Other features of the invention will become apparent in the course of the following descriptions of exemplary embodiments which are given for illustration of the invention and are not intended to be limiting thereof.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1.

### Improvement of Tumor Control by Combining a VEGF Trap and Radiation Therapy

U-87 cells, a clinically relevant human glioblastoma cell line, were injected subcutaneously into the right hind limb (5×10⁵ cells in 0.1 ml PBS) of athymic NCR NUM mice and allowed to grow until reaching a diameter of 4-5 mm before treatment. Tumor growth delay (TGD) was determined using time in days for the tumor to grow to 1000 mm³. In one experiment, a VEGF trap (SEQ ID NOs:3-4) was used at two doses, high (25 mg/kg) and low (2.5 mg/kg) given every three days for up to three weeks, using the same schedule with and without a single dose of radiation of 10 Grays (Gy).

In a second experiment, VEGF trap was used at either mid (10 mg/kg) or low (2.5 mg/kg) dose, and treatment was initiated one week prior to the single dose of radiation, following the radiation treatment, VEGF trap treatment was continued for an additional 21 days, again being administered every third day.

In a third experiment, VEGF trap was used at either mid (10 mg/kg) or low (2.5 mg/kg) dose, and treatment was initiated one week prior to fractionated radiation and continued for up to 21 days, being administered every third day. Fractionated radiation was given in 3 doses of 5 Gray each (3×5Gy) over three consecutive days (D7, 8 and 9). For tumors that received only fractionated radiation, tumors were size matched to those that received VEGF trap prior to radiation so that radiation therapy was administered to similarly sized tumors regardless of whether on not they had been pre-treated with VEGF trap.

Results: In the first study, control tumors had an average TGD of 10 days whereas low dose VEGF trap increased TGD an additional 10 days. A single dose of radiation of 10 Gy increased TGD 10 days over that of control whereas radiation plus low dose VEGF trap increased TGD 20-25 days over that of control. High dose VEGF trap increased TGD 40 days over that of control but did not show any increased benefit when combined with radiation. In the second study, when VEGF trap at either 10 mg/kg or 2.5 mg/kg was combined with radiation therapy, enhanced tumor suppression was observed. As seen in Table 1, average tumor size at an interim point in the study, Study Day 35, is reduced when VEGF trap (VEGFT) is combined with radiation. Additionally, fewer tumors reach the specified study endpoint by this time when combination therapy is given. The results show that suppression and delay in tumor growth is achieved by the combined treatments.

In the third study, average tumor growth in the untreated group corresponded to a doubling time of 3.1 days. Radiation alone when administered to tumors that were size matched to correspond to the smaller VEGF trap treated tumors, slowed tumor growth rate to approximately 5 days. The low-dose VEGF trap group (2.5 mg/kg) had a doubling time similar to that of radiation alone (4.8 days), while the high-dose VEGF trap group (10 mg/kg) lengthened doubling time (9.6 days; p=0.001 vs. control. Combination of low-dose VEGF trap and radiotherapy slowed the mean doubling time to 10.4 days, a stronger effect than that seen by a comparable regimen of VEGF trap alone (p=0.001), or radiation alone. The combination of high-dose VEGF trap with radiation slowed the mean tumor doubling time to 17.1 days, a stronger effect than that of either high-dose VEGF trap alone (p=0.070) or radiation alone.

It is concluded that VEGF trap alone is an effective inhibitor of tumor growth in the U-87 glioblastoma model and that low or mid dose VEGF trap in combination with radiation has an enhanced effect on tumor cell killing. These results have important implications for the treatment of human cancer.

TABLE 1
Study Day 35

| | Tumor Volume | # Mice Still |
|---|---|---|
| Treatment Group | (mm³) ± SEM | in Study |
| Control | 1854 ± 276 | 1/8 |
| Radiation Only | 2243 ± 104 | 0/8 |
| VEGFT 2.5 mg/kg | 1751 ± 174 | 1/9 |
| VEGFT 10 mg/kg | 1357 ± 205 | 6/10 |
| VEGFT 2.5 mg/kg + Radiation | 1400 ± 206 | 5/8 |
| VEGFT 10 mg/kg + Radiation | 668 ± 347 | 5/6 |

The present invention may be embodied in other specific forms without departing from the spirit or essential attributes thereof.

1. A method of treating cancer in a subject in need thereof, comprising administering to the subject a vascular endothelial growth factor (VEGF) antagonist in combination with radiation therapy such that the cancer is treated, wherein radiation therapy is administered as a single dose, a fractionated dose, or in multiple doses.

2. The method of claim 1, wherein the VEGF antagonist is VEGFR1R2-FcΔC1(a) or Flt1D2.Flk1D3.FcΔC1(a).

3. The method of claim 2, wherein the VEGF antagonist comprises the amino acid sequence of SEQ ID NO:1 or SEQ ID NO:3.

4. The method of claim 1, wherein administration of a VEGF antagonist and radiation is concurrent or sequentially.

5. The method of claim 4, wherein radiation is ionizing radiation therapy and/or a therapeutic radiopharmaceutical.

6. The method of claim 1, wherein the subject is a human subject.

7. The method of claim 3, wherein the VEGF antagonist is administered at a high dose of about 7.5 to 15 mg/kg.

8. The method of claim 3, wherein the VEGF antagonist is administered at a low dose of about 1 to 5 mg/kg.

9. The method of claim 1, wherein administration of the VEGF antagonist is subcutaneous, intramuscular, intradermal, intraperitoneal, intravenous, intranasal, epidural, or oral.

10. A method of reducing tumor growth in a subject in need thereof, comprising administering to the subject a vascular endothelial growth factor (VEGF) antagonist and radiation therapy, wherein the growth of the tumor is reduced.

11. The method of claim 10, wherein the VEGF antagonist is a VEGF trap selected from the group consisting of VEGFR1R2-FcΔC1(a) and Flt1D2.Flk1D3.FcΔC1(a).

12. The method of claim 11, wherein the VEGF trap comprises the amino acid sequence of SEQ ID NO:1 or SEQ ID NO:3.

13. The method of claim 10, wherein the VEGF antagonist is administered concurrently or sequentially with radiation therapy.

14. The method of claim 10, wherein the radiation therapy is ionizing radiation therapy and/or a therapeutic radiopharmaceutical.

15. A method of treating a human patient suffering from cancer, comprising administering an effective amount of a vascular endothelial growth factor (VEGF) antagonist and radiation to the human patient, the method comprising administering to the patient an initial dose of ≦5.0 mg/kg of the VEGF trap with radiation therapy.

16. The method of claim 15, wherein the VEGF antagonist is VEGFR1R2-FcΔC1(a) or Flt1D2.Flk1D3.FcΔC1(a).

17. The method of claim 15, wherein the VEGF antagonist is administered concurrently or sequentially with radiation therapy.

18. The method of claim 15, wherein the radiation therapy is ionizing radiation therapy and/or a therapeutic radiopharmaceutical.
